(19) United States
(12) Patent Application Publication
(10) Pub. No.: US 2023/0310935 A1
(43) Pub. Date: Oct. 5, 2023

(54) METHOD FOR DETERMINING EXERCISE PARAMETER BASED ON RELIABLE EXERCISE DATA

(71) Applicant: bOMDIC Inc., Taipei City (TW)

(72) Inventors: Yu Han Su, New Taipei City (TW); Yu Hsin-Ju, New Taipei City (TW); Yu Yu-Wei, New Taipei City (TW)

(21) Appl. No.: 17/709,851

(22) Filed: Mar. 31, 2022

(51) Int. Cl. A63B 24/00

(52) U.S. Cl. CPC A63B 24/0062; A63B 2024/0065; A63B 2024/0068

(57) ABSTRACT

The embodiments of the disclosure provide a method for determining an exercise parameter if the exercise data is reliable. The exercise data is reliable if the criterion set is met by the exercise data. The method comprises: acquiring exercise data; confirming whether a criterion set is met by a judgement parameter set determined based on the exercise data or not; and using the exercise data to determine an estimation of the exercise parameter if the criterion set is met by the judgement parameter set.

### FIELD OF THE INVENTION

The present invention relates to a method for determining an exercise parameter and, more particularly, to a method for determining an exercise parameter based on reliable exercise data.

### BACKGROUND

The exercise parameter (e.g., VO₂ₘₐₓ or FTP (Functional Threshold Power)) of the user of an exercise monitoring device must be precisely estimated before an optimized exercise guidance can be provided for the user to improve their fitness or health of the user.

Conventionally, the exercise parameter is estimated based on exercise data (e.g., heart rate or speed/power) using a sensing unit in the workout. However, inaccurate/unreliable exercise data are usually acquired in some conditions; for example, the measuring device (e.g., wearable device) is not fully fastened to the skin, or the measuring device is operating abnormally. The inaccurate/unreliable exercise data may lead to the imprecision of the estimated exercise parameter of the user.

Accordingly, there is a benefit to improving the determination of an exercise parameter to overcome the above-mentioned disadvantages.

### SUMMARY OF THE INVENTION

The present invention discloses a method for determining if an acquired exercise data is reliable and then determining an exercise parameter when the exercise data is reliable. The exercise data is deemed reliable if a criterion set is met by the exercise data. The method comprises: acquiring exercise data; confirming whether a criterion set is met by a judgement parameter set determined based on the exercise data or not; and using the exercise data to determine an estimation of the exercise parameter if the criterion set is met by the judgement parameter set.

By the algorithm implemented in the computer of the present invention, the computer of the present invention performs operations described in claims or the following descriptions to determine an exercise parameter.

The detailed technology and above preferred embodiments implemented for the present invention are described in the following paragraphs accompanying the appended drawings for people skilled in the art to well appreciate the features of the claimed invention.

### BRIEF DESCRIPTION OF THE DRAWINGS

The foregoing aspects and many of the accompanying advantages of this invention will become more readily appreciated as the same becomes better understood by reference to the following detailed description when taken in conjunction with the accompanying drawings, wherein:

FIG. 1 illustrates a schematic block diagram of an exemplary apparatus in the present invention;

FIG. 2 illustrates a method for determining an exercise parameter if the exercise data is reliable;

FIG. 3 illustrates an embodiment of a content of a criterion set of FIG. 2;

FIG. 4A to FIG. 4D illustrate example conditions of the consistency between a first trend of the first internal workload data subset and a second trend of the first external workload data subset in the first duration when changing an exercise intensity;

FIG. 5A to FIG. 5D illustrate example conditions of an extent to which the first internal workload data subset follows the first external workload data subset in the first duration when changing an exercise intensity; and

FIG. 6 illustrates a precision of an estimation of the exercise parameter being VO₂ₘₐₓ.

### DETAILED DESCRIPTION OF THE ILLUSTRATED EMBODIMENTS

The detailed explanation of the present invention is described as following. The described embodiments are presented for purposes of illustrations and description and they are not intended to limit the scope of the present invention.

Exercise Data

The exercise data is acquired by using a sensing unit 11 when the user performs an exercise (physical exercise) in an exercise session. The exercise data may comprise at least one of (i) the internal workload data (internal workload data set) associated with the internal workload and (ii) the external workload data associated with the external workload. The exercise data may also comprise the internal workload data (internal workload data set) associated with the internal workload and the external workload data (external workload data set) associated with the external workload.

External Workload

The data of the external workload may refer to data acquired during training completed by the user and is generated from sensors placed outside of the body and measured independently of the user's internal characteristics.

Internal Workload

The data of the internal workload may refer to the relative physiological and psychological stress imposed by the external workload and is generated as a representation, indication, and/or state of the inside operation of the body. The internal workload is associated with the user's internal characteristics. The external workload has a different effect on the internal workload among the users. The acquired training result may be employed as an association with the interaction between the internal workload and the external workload.

Exercise Intensity

The data of the exercise intensity may refer to how much energy is expended when a user is undertaking an activity. The exercise intensity may define how hard the body has to work to overcome an activity/exercise. The exercise intensity may be measured in the form of the internal workload. The parameter of the exercise intensity associated with the internal workload may be associated with heart rate, oxygen consumption, pulse, respiration rate, and RPE (rating perceived exertion). The exercise intensity may be measured in the form of the external workload. The parameter of the exercise intensity associated with the external workload may be associated with speed, acceleration, power, force, energy expenditure rate, motion intensity, a motion cadence, or other kinetic data created by the external workload resulting in energy expenditure. The heart rate may be often used as a parameter of the exercise intensity.

Criterion Set

An example of a criterion set 24 are provided in FIG. 3. To acquire the reliable exercise data for determining the exercise parameter, the present invention sets up the criterion set 24 for confirming whether the exercise data is reliable or not. The criterion set 24 may include a first criterion (i), a second criterion (ii), and so on.

Judgement Parameter Set

An example of a judgement parameter set 25 is provided in FIG. 3. The judgement parameter set 25, and associated value(s), are associated with or refer to an indication of the reliability in an estimation of the exercise parameter. The judgement parameter set 25 can be defined and used in the criterion set 24. If the criterion set 24 is met based on (e.g., at least one value of) the judgement parameter set 25 (and the criterion set 24 is met if all of the criteria in the criterion set 24 are met), the exercise data is deemed reliable for determining the exercise parameter. The judgement parameter set 25 may include a first judgement parameter J1, a second judgement parameter J2, and so on.

Feature Parameter Set

An example of a feature parameter set 26 is provided in FIG. 3. The feature parameter set 26, and associated value(s), may be derived from the exercise data. The parameter(s) and associated values in the judgement parameter set 25 may be determined based on the parameter(s) in the feature parameter set 26. The parameter(s) in the feature parameter set 26 may be associated with or refers to an indication of the reliability in an estimation of the exercise parameter and may be used as the parameter(s) in the judgement parameter set 25. The feature parameter set 26 may include a first feature parameter F1, a second feature parameter F2, a third feature parameter F3, and so on.

The method in the present invention may be applied in all kinds of apparatuses, such as a measurement system that is worn on the individual (e.g., the device attached to the wrist band or chest belt), a wrist-top device, a mobile device, a portable device, a personal computer, a server or a combination thereof.

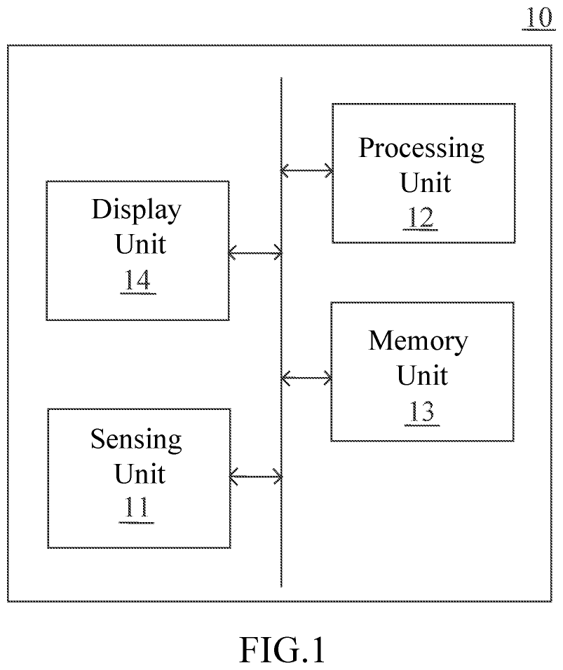

FIG. 1 illustrates a schematic block diagram of an exemplary apparatus 10 in the present invention. The apparatus 10 may comprise a sensing unit 11, a processing unit 12, a memory unit 13, and a displaying unit 14. The units of the apparatus 10 may communicate with another unit in a wired or wireless manner. The sensing unit 11 may be in one device (e.g., the device worn on the individual or watch), and the processing unit 12 may be in another device (e.g., mobile device or mobile phone). Alternatively, the sensing unit 11 and the processing unit 12 may be in a single device (e.g., the device worn on the individual or watch). The sensing unit 11 may be attached to or built into a belt worn on the individual. The sensing unit 11 may be a sensor (e.g., heart activity sensor) that may measure a signal associated with the physiological data, the cardiovascular data, or the internal workload of the person's body. The signal may be measured when the sensor unit 11 is in skin contact with the chest, wrist, or other human part. The processing unit 12 may be any suitable processing device for executing software instructions, such as a central processing unit (CPU). The processing unit 12 may be a computing unit.

The apparatus 10 may comprise at least one device; a first portion of the computing unit may be in one device (e.g., the device worn on the individual or watch), a second portion of the computing unit may be in another device (e.g., mobile device or mobile phone) and a first portion of the computing unit may communicate with a second portion of the computing unit in a wired or wireless way; a first portion of the computing unit and a second portion of the computing unit may be in a single device (e.g., the device worn on the individual or watch). The memory unit 13 may include random access memory (RAM) and read-only memory (ROM), but it is not limited to this case. The memory unit 13 may include any suitable non-transitory computer-readable medium, such as ROM, CD-ROM, DVD-ROM, and so on. Also, the non-transitory computer-readable medium is a tangible medium. The non-transitory computer-readable medium includes a computer program code which, when executed by the processing unit 12, causes the apparatus 10 to perform desired operations (e.g., operations listed in claims). The display unit 14 may be a display for displaying an estimation of the exercise parameter. Optionally, the first reference value of the first physiological parameter and the second reference value of the second physiological parameter is also displayed. The displaying mode may be in the form of words, a voice, or an image. The sensing unit 11, the processing unit 12, the memory unit 13, and the displaying unit 14 in the apparatus 10 may have any suitable configuration, and it doesn't be described in detail therein.

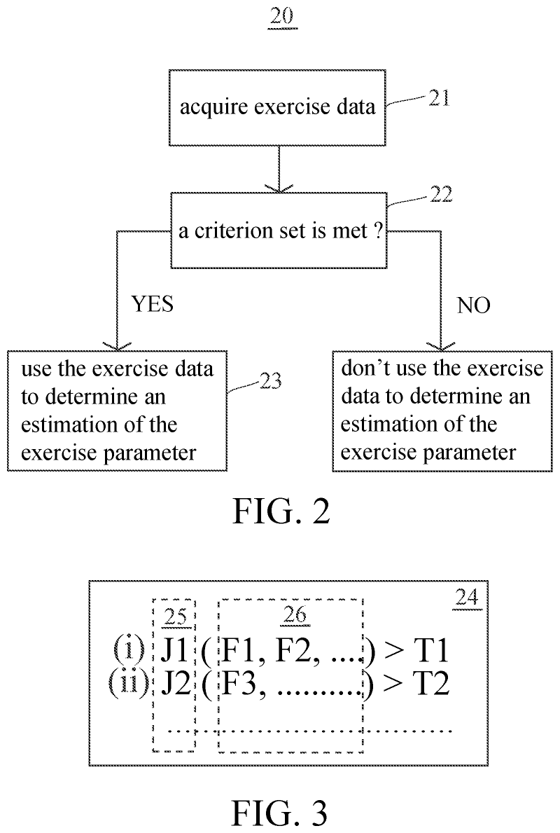

FIG. 2 illustrates a method 20 for determining an exercise parameter if the exercise data is deemed reliable. The exercise data is deemed reliable if the criterion set 24 is met through an analysis of the exercise data, i.e., the criterion set 24 is met if all the criteria in the criterion set 24 are met. The method comprises:

Step 21: acquire exercise data;

Step 22: confirm whether a criterion set is met by a judgement parameter set determined based on the exercise data or not;

Step 23: use the exercise data to determine an estimation of the exercise parameter if the criterion set is met by the judgement parameter set.

### Embodiment (A)

When the user performs an exercise in an exercise session, the user may adopt an exercise routine or exercise mode of operation comprising: (Type 1) change an exercise intensity to a larger extent and (Type 2) keep a constant exercise intensity or keep an exercise intensity within a range. In Type “1” routine or operation, a variance of the exercise intensity may be higher than a variance threshold TA1, which can be assessed in the system and corresponding algorithm of the present invention. In Type “2” routine or operation, the variance of the exercise intensity may be lower than a variance threshold TA2, which can also be assessed in the system and corresponding algorithm of the present invention. Because the exercise data in Type 1 is more complex than that in Type 2, and the deviation between the internal workload data and the external workload data in Type 1 mode may be higher than that in Type 2, EMBODIMENT (A) of the present invention focuses on performing the algorithm mainly on the exercise data in Type 1 to acquire the reliable exercise data for determining the exercise parameter.

The acquired exercise data in an exercise session may comprise an internal workload data set and an external workload data set (in step 21). The internal workload data set is temporally corresponding to the external workload data set in being acquired simultaneously or contemporaneously to one another. The internal workload data set may include a first parameter associated with an exercise intensity. The first parameter of the exercise intensity may comprise heart rate, oxygen consumption, pulse, respiration rate, and RPE (rating perceived exertion). Preferably, the first parameter of the exercise intensity is heart rate. The external workload data set may include a second parameter associated with the exercise intensity. The second parameter of the exercise intensity may comprise speed, acceleration, power, force, energy expenditure rate, motion intensity, a motion cadence, or other kinetic data created by the external workload resulting in energy expenditure. Preferably, the second parameter is the measured speed of the user acquired during a running exercise or the power level (e.g., measured or estimated power or power level) acquired during a cycling exercise.

The internal workload data set and the external workload data set may be acquired by using a sensing unit 11. In one embodiment, the internal workload data set may be measured by a first sensor of the sensing unit 11, and the external workload data may be measured by a second sensor of the sensing unit 11. The first sensor may be different from the second sensor. For example, the internal workload data set is heart activity data, and the first sensor is heart activity sensor; the external workload data is motion data, and the second sensor is a motion sensor. Each/one of the internal workload data set and the external workload data set may be derived from raw data measured by a corresponding sensor.

The exercise session may comprise a first duration when Type 1 is adopted. The first duration may be a continuous duration or a total duration of many minor durations. The adjacent minor durations have an interval therebetween. The internal workload data set comprises a first internal workload data subset in the first duration and the external workload data set comprises a first external workload data subset in the first duration (i.e., the first internal workload data subset temporally corresponding to the first external workload data subset). In the first duration when Type 1 is adopted, a variance of at least one of the first internal workload data subset and the first external workload data subset may be higher than a variance threshold TB. In a first example, a variance of the first internal workload data may be higher than a variance threshold TB1; in a second example, a variance of the first external workload data may be higher than a variance threshold TB2; in a third example, a variance of the first internal workload data may be higher than a variance threshold TB3, and a variance of the first external workload data may be higher than a variance threshold TB4.

Because the generated internal workload (e.g., heart rate) has a time-delay effect when the external workload (e.g., speed) is generated, the first external workload data subset (e.g., speed) may be determined by modifying a first initial internal workload data subset (e.g., initial speed) such that the first external workload data subset (e.g., speed) synchronizes with the first internal workload data subset (e.g., heart rate) more than the first initial internal workload data subset (e.g., initial speed). The first initial internal workload data subset (e.g., initial speed) may be modified by any suitable method, such as a moving-average method.

To acquire reliable exercise data for determining the exercise parameter, the present invention sets up a criterion set 24 for confirming whether the exercise data is reliable or not (step 22). The criterion set may comprise at least one criterion subset or at least one criterion. FIG. 3 illustrates an embodiment of a content of a criterion set 24 in step 22 of FIG. 2. A judgement parameter set 25 (e.g., the parameters J1, J2, . . . in FIG. 3) associated with the reliability in estimating the exercise parameter can be defined and used in the criterion set 24. If the criterion set 24 is met by at least one value of the judgement parameter set 25, i.e., the criterion set 24 is met if all the criteria in the criterion set 24 are met, and the exercise data is deemed reliable for determining the exercise parameter. Further, high precision of an estimation of the judgement parameter set 25 can precisely determine and/or indicate whether the exercise data is reliable or not for further determining the exercise parameter. Therefore, to increase the precision of the estimation of the judgement parameter set 25, the present invention determines the judgement parameter set 25 based on a first feature parameter (refer to one of the parameters F1, F2, F3 in the feature parameter set 26 in FIG. 3) being consistent, or having consistency, between a first trend of the first internal workload data subset and a second trend of the first external workload data subset in the first duration when Type 1 is adopted.

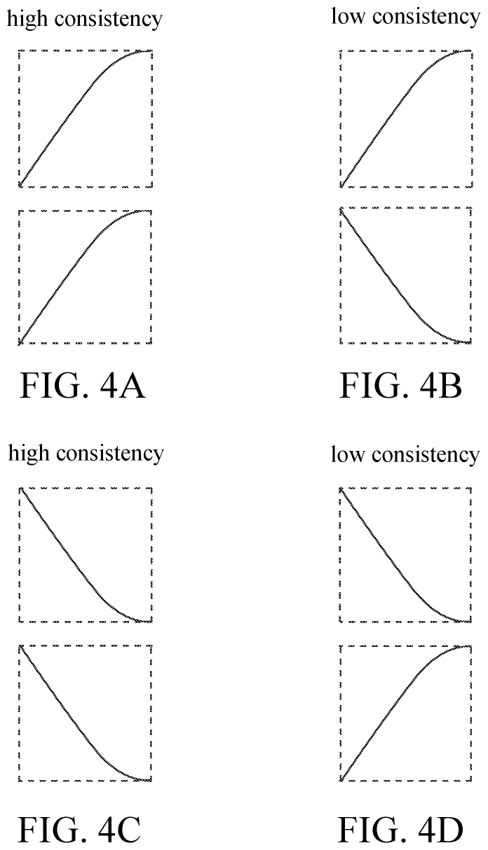

FIG. 4A to FIG. 4D illustrate some conditions of the consistency between a first trend of the first internal workload data subset and a second trend of the first external workload data subset in the first duration when Type 1 is adopted. Each of the first trend of the first internal workload data subset and the second trend of the first external workload data subset may be an increasing trend of the corresponding exercise intensity varying with time or a decreasing trend of the corresponding exercise intensity varying with time. For the convenience of description, the top portion of each of FIG. 4A to FIG. 4D only illustrates a portion of the first internal workload data subset and the bottom portion of each of FIG. 4A to FIG. 4D only illustrates the corresponding portion of the first external workload data subset. Each of the left terminal and the right terminal of each curve of FIG. 4A to FIG. 4D is a relatively high point or a relatively low point. In FIG. 4A, each of the first trends of the first internal workload data subset and the second trend of the first external workload data subset is an increasing trend of the corresponding exercise intensity varying with time, so the trend consistency is high. In FIG. 4C, each of the first trends of the first internal workload data subset and the second trend of the first external workload data subset is a decreasing trend of the corresponding exercise intensity varying with time, so the trend consistency is high. In FIG. 4B and FIG. 4D, the first trend of the first internal workload data subset is different from the second trend of the first external workload data subset, so the trend consistency is low.

The greater the consistency between the first trend of the first internal workload data subset and the second trend of the first external workload data subset in the first duration when Type 1 is adopted is, the less the internal workload data deviates from the external workload data (e.g., shown in FIG. 4A to FIG. 4D). Because the consistency is associated with or determined based on an assessed metric for the deviation, the precision of the estimation of the judgement parameter set 25 associated with reliability in an estimation of the exercise parameter can be improved by using the first feature parameter.

In one embodiment, the first feature parameter is a correlation degree (e.g., correlation coefficient) between the first internal workload data subset and the first external workload data subset in the first duration when Type 1 is adopted.

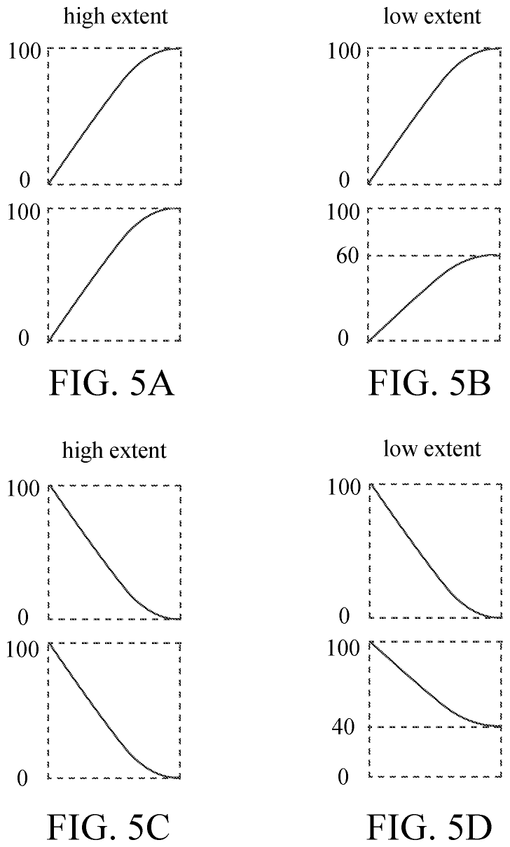

In order to further increase the precision of an estimation of the judgement parameter set or precisely judge whether the estimated first feature parameter is reliable or not, the present invention determines the judgement parameter set based on a second feature parameter (refer to one of the parameters F1, F2, F3 in the feature parameter set 26 in FIG. 3) being an extent to which the first internal workload data subset follows (in proximity to) the first external the workload data subset in the first duration when Type 1 is adopted. Generally, it is meaningful that the first internal workload data subset follows the first external workload data subset, which can be determined if the consistency between the first trend of the first internal workload data subset and the second trend of the first external workload data subset is sufficiently high. So, the first internal workload data subset and the first external workload data subset, shown in FIG. 4A and FIG. 4C, has a priority to determine its extent to which the first internal workload data subset follows the first external workload data subset because of the high trend consistency, which will be described in detail in FIG. 5A to FIG. 5D. Preferably, the present invention determines the judgement parameter set 25 based on a combination of the first feature parameter and the second feature parameter if the second feature parameter is taken into account in determining the judgement parameter set 25.

FIG. 5A to FIG. 5D illustrate example conditions of an extent to which the first internal workload data subset follows the first external workload data subset in the first duration when Type 1 is adopted. For the convenience of description, the top portion of each of FIG. 5A to FIG. 5D only illustrates a portion of the first internal workload data subset and the bottom portion of each of FIG. 5A to FIG. 5D only illustrates the (temporally) corresponding portion of the first external workload data subset. Each of the left terminal and the right terminal of each curve of FIG. 5A to FIG. 5D is a relatively high point or a relatively low point. The number in the vertical axis represents the normalized exercise extensity. In FIG. 5A, each of the first trends of the first internal workload data subset and the second trend of the first external workload data subset is an increasing trend of the corresponding exercise intensity varying with time and has the same increment of the normalized exercise extensity, so the following extent is high. In FIG. 5C, each of the first trends of the first internal workload data subset and the second trend of the first external workload data subset is a decreasing trend of the corresponding exercise intensity varying with time and has the same decrement of the normalized exercise extensity, so the following extent is high. In FIG. 5B, each of the first trends of the first internal workload data subset and the second trend of the first external workload data subset is an increasing trend of the corresponding exercise intensity varying with time and has a different increment of the normalized exercise extensity, so the following extent is low. In FIG. 5D, each of the first trends of the first internal workload data subset and the second trend of the first external workload data subset is a decreasing trend of the corresponding exercise intensity varying with time and has a different decrement of the normalized exercise extensity, so the following extent is low.

The more the extent to which the first internal workload data subset follows the first external workload data subset in the first duration when Type 1 is adopted is, the less the internal workload data deviates from the external workload data. Because the extent is associated with the deviation, the precision of the estimation of the judgement parameter set 25 associated with the reliability in an estimation of the exercise parameter can be improved by using the second feature parameter or whether the estimated first feature parameter is reliable or not can be precisely judged by using the second feature parameter.

In one embodiment, the second feature parameter is a slope in the regression analysis (e.g., linear regression) of the first internal workload data subset and the first external workload data subset in the first duration when Type 1 is adopted.

To further increase the precision of an estimation of the judgement parameter set 25, the present invention determines the judgement parameter set 25 based on a third feature parameter (refer to one of the parameters F1, F2, F3 in the feature parameter set 26 in FIG. 3) being a duration length of the first duration when the first internal workload data subset and the first external workload data subset are acquired in the first duration when Type 1 is adopted. Preferably, the present invention determines the judgement parameter set 25 based on a combination of the first feature parameter, the second feature parameter, and the third feature parameter. Preferably, the present invention determines the judgement parameter set 25 based on a combination of the first feature parameter, the second feature parameter, and the third feature parameter if the third feature parameter is taken into account in determining the judgement parameter set 25.

The exercise session may comprise a second duration when Type 2 is adopted. The second duration may be a continuous duration or a total duration of many minor durations. The adjacent minor durations have an interval therebetween. The internal workload data set comprises a second internal workload data subset in the second duration and the external workload data set comprises a second external workload data subset in the second duration (i.e., the second internal workload data subset is temporally corresponding to the second external workload data subset). In the second duration, when Type 2 is adopted, a variance of one of at least one of the second internal workload data subset and the second external workload data subset may be lower than a second variance threshold TC. In a first example, a variance of the second internal workload data may be higher than TC1; in a second example, a variance of the second external workload data may be higher than TC2; in a third example, a variance of the first internal workload data may be higher than TC3 and a variance of the first external workload data may be higher than TC4.

The judgement parameter set 25 may be determined based on any suitable feature parameter(s) (refer to the parameters F1, F2, F3 in the feature parameter set in FIG. 3). In one embodiment, the exercise data in Type 2 may be used in the algorithm to acquire the reliable exercise data for determining the exercise parameter. The feature parameter may be associated with the second internal workload data subset and the second external workload data subset. For example, the feature parameter is the error (e.g., mean error) between the data (including the second internal workload data subset and the second external workload data subset in the second duration when Type 2 is adopted) and the regression line in the regression analysis (e.g., linear regression) of the data. The feature parameter may be a duration length of the second duration when the second internal workload data subset and the second external workload data subset are acquired in the second duration when Type 2 is adopted.

The exercise data is used to determine an estimation of the exercise parameter if the result “the criterion set is met by the judgement parameter set” is YES (step 23). The exercise parameter may be calculated based on the exercise data. Specifically, the exercise data may comprise a first portion of the exercise data that meets the criterion set 24 (i.e. the judgement parameter set 25 determined based on a first portion of the exercise data meets the criterion set 24) and a second portion of the exercise data which doesn't meet the criterion set 24 (i.e. the judgement parameter set 25 determined based on a second portion of the exercise data doesn't meet the criterion set 24); the exercise parameter can be calculated based on a first portion of the exercise data which meets the criterion set 24 (not based on a second portion of the exercise data which doesn't meet the criterion set 24). The exercise parameter may be calculated based on at least one of the first internal workload data subset and the first external workload data subset. In a first example, the exercise parameter may be calculated based on the first internal workload data subset; in a second example, the exercise parameter may be calculated based on the first external workload data subset; in a third example, the exercise parameter may be calculated based a combination of the first internal workload data subset and the first external workload data subset. The exercise parameter may be calculated based on at least one of the internal workload data set and the external workload data set. In a first example, the exercise parameter may be calculated based on the internal workload data set; in a second example, the exercise parameter may be calculated based on the external workload data set; in a third example, the exercise parameter may be calculated based a combination of the first internal workload data set and the first external workload data subset. On the contrary, the exercise data is not used to determine an estimation of the exercise parameter if the result “the criterion set is met by the judgement parameter set” is NO.

Determining an estimation of the exercise parameter may comprise (1) calculating the exercise parameter based on at least one of the first internal workload data subset and the first external workload data subset after confirming that the criterion set 24 is met by the judgement parameter set 25 (i.e., the result is YES in step 23); and (2) calculating the exercise parameter based on at least one of the first internal workload data subset and the first external workload data before confirming whether the criterion set 24 is met by a judgement parameter set 25 or not and then keep the exercise parameter calculated based on at least one of the first internal workload data subset and the first external workload data subset after confirming that the criterion set 24 is met by the judgement parameter set 25 (i.e. the result is YES in step 23). After determining the estimation of the exercise parameter, the estimation of the exercise parameter may be displayed by a displaying unit 14 and/or may be processed to generate the next/advanced exercise parameter.

The exercise parameter in EMBODIMENT (A) may be an energy expenditure, a fitness performance level (the fitness performance level may include health-related fitness and sport/skill-related fitness, which can also be improved by engaging in physical activities or training, e.g., VO₂ₘₐₓ or FTP (Functional Threshold Power)), first lactate threshold (LT1), second lactate threshold (LT2), maximum heart rate (HRmax) or minimum heart rate (HRmin), training load, fatigue, training effect, recovery, stamina. The exercise parameter may be calculated by any suitable method. For example, stamina and energy expenditure may be determined by taking the U.S. application Ser. No. 14/718,104, U.S. application Ser. No. 17/070,040, U.S. application Ser. No. 17/070,947 as the references; the maximum heart rate may be determined by taking the U.S. application Ser. No. 17/376,146 as a reference; the fitness performance level (e.g., VO₂ₘₐₓ or FTP (Functional Threshold Power) may be determined based on the maximum heart activity parameter (e.g., maximum heart rate (HRmax)) by any suitable method, such as a combination of the maximum heart activity parameter and a statistics of the internal workload data and the external workload data.

To acquire the reliable exercise data for determining the exercise parameter, a criterion set 24 may have any suitable content for confirming whether the exercise data is reliable or not (step 22).

### Embodiment (A-1)

In one embodiment of the criterion set, the criterion set 24 comprises a first criterion describing that the first judgement parameter of the judgement parameter set 25 is higher than a reliability threshold, and the first judgement parameter of the judgement parameter set 25 is the reliability in the estimation of the exercise parameter. The reliability in the estimation of the exercise parameter may be determined based on a first feature parameter (i.e., the consistency between a first trend of the first internal workload data subset and a second trend of the first external workload data subset).

The reliability in the estimation of the exercise parameter may be determined further based on the second feature parameter (i.e., the extent to which the first internal workload data subset follows the first external workload data subset). Preferably, the reliability in the estimation of the exercise parameter is determined based on a combination of the first feature parameter and the second feature parameter.

The following algorithm is a first example of determining the reliability in the estimation of the exercise parameter; however, the present invention is not limited to this case.

R(F1,F2)=c1*F1+c2*F2+any other suitable term (1)

In a prefer embodiment, R (F1, F2)=c1*F1+c2*F2

R is the reliability in the estimation of the exercise parameter (i.e., the first judgement parameter of the judgement parameter set 25), F1 is the consistency between the first trend of the first internal workload data subset and the second trend of the first external workload data subset (i.e., first feature parameter), F2 is the extent to which the first internal workload data subset follows the first external workload data subset. (i.e., the second feature parameter), each of c1 to c2 is a coefficient adjusted according to the observation of the physiological phenomenon.

The following algorithm is a second example of determining the reliability in estimating the exercise parameter; however, the present invention is not limited to this case.

R(F1,F2)=c1*F1+any other suitable term, if F2>THQ (2)

In a prefer embodiment, R (F1, F2)=c1*F1, if F2>THQ

R is the reliability in the estimation of the exercise parameter (i.e., the first judgement parameter of the judgement parameter set), F1 is the consistency between the first trend of the first internal workload data subset and the second trend of the first external workload data subset (i.e., first feature parameter), F2 is the extent to which the first internal workload data subset follows the first external workload data subset. (i.e., the second feature parameter), THQ is a threshold of F2, which is used for judging whether the estimated first feature parameter is reliable or not, c1 is a coefficient adjusted according to the observation of the physiological phenomenon.

### Embodiment (A-2)

In one embodiment of the criterion set 24, the criterion set 24 comprises a first criterion that describes that the first judgement parameter of the judgement parameter set 25 is higher than a consistency threshold, and the first judgement parameter of the judgement parameter set 25 is the first feature parameter (i.e., the consistency between the first trend of the first internal workload data subset and the second trend of the first external workload data subset).

The criterion set 24 may further comprise a second criterion describes that the second judgement parameter of the judgement parameter set 25 is higher than an extent threshold, and the second feature parameter of the judgement parameter set 25 is the second feature parameter (i.e., the extent to which the first internal workload data subset follows the first external workload data subset).

EXPERIMENT RESULT of EMBODIMENT (A)

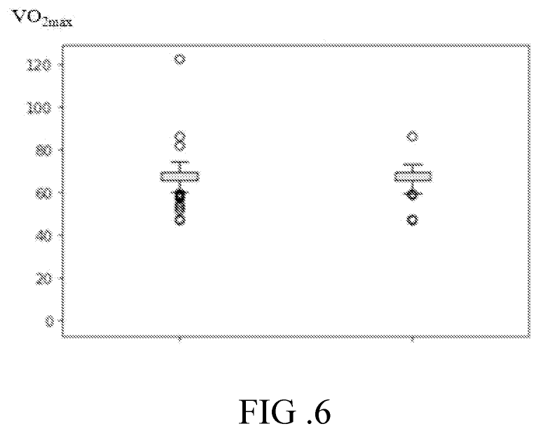

FIG. 6 illustrates the precision of estimating the exercise parameter being VO2max. The left portion is a distribution of VO2max for the user without using the method in the present invention. The right portion is a distribution of VO2max for the user by using the method in the present invention. As shown, the distribution of VO2max is narrowed to increase the precision of estimation of VO2max.

### Embodiment (B)

EMBODIMENT (B) of the present invention focuses on performing an algorithm mainly on the exercise data with the substantially/gradually increasing exercise intensity to acquire the reliable exercise data for determining the exercise parameter. The exercise data with the substantially/gradually increasing exercise intensity may mean that the exercise intensity of most of the exercise data in a duration gradually increases but the exercise intensity of a small portion of the exercise data in the duration decreases. The exercise data with the substantially/gradually increasing exercise intensity can be used for increasing the precision of an estimation of the exercise parameter associated with the heavy exercise.

### Embodiment (B-1)

The exercise data is acquired by using a sensing unit 11 in an exercise session (in step 21). The exercise data may use a first parameter of exercise intensity. The first parameter of the exercise intensity associated with the internal workload data set may comprise heart rate, oxygen consumption, pulse, respiration rate, and RPE (rating perceived exertion). Preferably, the first parameter of the exercise intensity associated with the internal workload is heart rate. The first parameter of the exercise intensity associated with the external workload may comprise speed, acceleration, power, force, energy expenditure rate, motion intensity, a motion cadence, or other kinetic data created by the external workload resulting in energy expenditure. Preferably, the first parameter of the exercise intensity associated with the external workload is speed. Preferably, the first parameter of the exercise intensity associated with the external workload is power. More preferably, the first parameter of the exercise intensity associated with the external workload is the measured speed in a running exercise, or the first parameter of the exercise intensity associated with the external workload is a power (e.g., measured or estimated power or power level) in a cycling exercise. A sensor used for acquiring the exercise data depends on the first parameter of the exercise intensity used in the exercise data; for example, the exercise data set is heart activity data, and the first sensor is heart activity sensor; the external workload data is motion data and the second sensor is a motion sensor. The exercise data may be derived from raw data measured by a corresponding sensor.

To acquire the reliable exercise data for determining the exercise parameter, the present invention sets up a criterion set 24 for confirming whether the exercise data is reliable or not (step 22). The criterion set may comprise at least one criterion subset or at least one criterion. FIG. 3 illustrates an embodiment of the content of a criterion set 24 in step 22 of FIG. 2. A judgement parameter set 25 (e.g., the parameters J1, J2, . . . in FIG. 3) associated with the reliability in an estimation of the exercise parameter can be defined and used in the criterion set 24. If the criterion set 24 is met by the judgement parameter set 25 (i.e., the criterion set 24 is met if all of the criteria in the criterion set 24 are met), the exercise data is deemed reliable for determining the exercise parameter. The criterion set 24 comprises a comparison between the judgement parameter set 25 and the corresponding threshold to judge whether the exercise data is reliable or not, so a high precision of the corresponding threshold of the judgement parameter set 25 can precisely judge whether the exercise data is reliable or not for further determining the exercise parameter. Therefore, to increase the precision of the corresponding threshold of the judgement parameter set 25, the corresponding threshold of the judgement parameter set 25 is associated with a first historical record of the judgement parameter set 25 in the present invention.

In one embodiment of the criterion set 24, the criterion set 24 comprises a first criterion describing that the first judgement parameter of the judgement parameter set 25 is higher than a first intensity threshold and the first judgement parameter of the judgement parameter set 25 is the first parameter of the exercise intensity. The first intensity threshold may be associated with a first historical record of the first parameter of the exercise intensity. In one embodiment, the first intensity threshold is determined based on a first statistic of the first parameter of the exercise intensity; for example, the first intensity threshold is a first statistic value (e.g., mean value or median value) of the first parameter of the exercise intensity. If the first parameter of the exercise intensity is higher than the first intensity threshold associated with the first historical record of the first parameter of the exercise intensity, the exercise data can have the substantially/gradually increasing exercise intensity, and thus EMBODIMENT (B) of the present invention can focus on performing an algorithm mainly on the exercise data with the substantially/gradually increasing exercise intensity to acquire the reliable exercise data for determining the exercise parameter. Optionally, the criterion set 24 may comprise any other criterion different from the first criterion; for example, the first parameter of the exercise intensity is higher than a first constant intensity threshold; this criterion may confirm that the user is performing an exercise (e.g., heavy exercise) to further precisely judge whether the exercise data is reliable or not for further determining the exercise parameter.

The exercise data is used to determine an estimation of the exercise parameter if the result “the criterion set is met by the judgement parameter set” is YES (step 23). The exercise parameter may be calculated based on the exercise data. Specifically, the exercise data may comprise a first portion of the exercise data that meets the criterion set 24 (i.e., the judgement parameter set 25 determined based on a first portion of the exercise data meets the criterion set 24) and a second portion of the exercise data that doesn't meet the criterion set 24 (i.e., the judgement parameter set 25 determined based on a second portion of the exercise data doesn't meet the criterion set 24); the exercise parameter can be calculated based on a first portion of the exercise data which meets the criterion set 24 (not based on a second portion of the exercise data which doesn't meet the criterion set 24). On the contrary, the exercise data is not used to determine an estimation of the exercise parameter if the result “the criterion set is met by the judgement parameter set” is NO.

Determining an estimation of the exercise parameter may comprise (1) calculating the exercise parameter based on the exercise data after confirming that the criterion set 24 is met by the judgement parameter set 25 (i.e. the result is YES in step 23); and (2) calculating the exercise parameter based on the exercise data before confirming whether the criterion set 24 is met by a judgement parameter set 25 or not then keep the exercise parameter calculated based on the exercise data after confirming that the criterion set 24 is met by the judgement parameter set 25 (i.e., the result is YES in step 23). After determining the estimation of the exercise parameter, the estimation of the exercise parameter may be displayed by a displaying unit 14 or the estimation of the exercise parameter or may be processed to generate the next/advance exercise parameter.

### Embodiment (B-2)

The acquired exercise data in an exercise session may comprise internal workload data and external workload data (in step 21). The internal workload data is temporally corresponding to the external workload data. The internal workload data set may include or be derived from a first parameter of exercise intensity. The first parameter of the exercise intensity may comprise heart rate, oxygen consumption, pulse, respiration rate, and RPE (rating perceived exertion). Preferably, the first parameter of the exercise intensity is heart rate. The external workload data may include or be derived from a second parameter of the exercise intensity. The second parameter of the exercise intensity may comprise speed, acceleration, power, force, energy expenditure rate, motion intensity, motion cadence, or other kinetic data created by the external workload resulting in energy expenditure. Preferably, the second parameter of the exercise intensity is speed. Preferably, the second parameter of the exercise intensity is power. More preferably, the second parameter of the exercise intensity is the measured speed in a running exercise, and the second parameter of the exercise intensity is the power (e.g., measured or estimated power or power level) in a cycling exercise.

The internal workload data set and the external workload data set may be acquired by using a sensing unit 11. In one embodiment, the internal workload data set may be measured by a first sensor of the sensing unit 11, and the external workload data may be measured by a second sensor of the sensing unit 11. The first sensor may be different from the second sensor. For example, the internal workload data set is heart activity data, and the first sensor is heart activity sensor; the external workload data is motion data, and the second sensor is a motion sensor. Each/one of the internal workload data set and the external workload data may be derived from raw data measured by a corresponding sensor.

The criterion set 24 of EMBODIMENT (B-1) may further comprise a second criterion that describes that the second judgement parameter of the judgement parameter set 25 is higher than a second intensity threshold in which the second feature parameter of the judgement parameter set 25 is the second parameter of the exercise intensity. In other words, the internal workload data set of the exercise data includes the first parameter of the exercise intensity (corresponding to the first parameter of the exercise intensity associated with the internal workload in EMBODIMENT (B-1)), and the external workload data includes the second parameter of the exercise intensity. The second intensity threshold may be associated with a second history record of the second parameter of the exercise intensity. In one embodiment, the second intensity threshold is determined based on a second statistic of the second parameter of the exercise intensity; for example, the second intensity threshold is a second statistic value (e.g., mean value or median value) of the second parameter of the exercise intensity. If the second parameter of the exercise intensity is higher than the second intensity threshold associated with the second history record of the second parameter of the exercise intensity, the exercise data can have the substantially/gradually increasing exercise intensity, and thus EMBODIMENT (B) of the present invention can focus on performing an algorithm mainly on the exercise data with the substantially increasing exercise intensity to acquire the reliable exercise data for determining the exercise parameter. Optionally, the criterion set 24 may comprise any other criterion different from the second criterion; for example, the second parameter of the exercise intensity is higher than a second constant intensity threshold; this criterion may confirm that the user is performing an exercise (e.g., heavy exercise) to further precisely judge whether the exercise data is reliable or not for further determining the exercise parameter.

In one embodiment, the judgement parameter set comprises a third judgement parameter determined based on a first feature parameter being a deviation degree between the internal workload data and the external workload data. The third judgement parameter is the deviation degree between the internal workload data and the external workload data, and the criterion set 25 comprises a comparison between the third judgement parameter and a deviation threshold of the third judgement parameter. For example, the deviation degree is represented in the form of a correlation degree (e.g., correlation coefficient), and the exercise data is reliable for determining the exercise parameter if the correlation degree is higher than a correlation threshold. For example, the deviation degree is represented in the form of the error (e.g., mean error) between the data (including the internal workload data and the external workload data) and the regression line in the regression analysis (e.g., linear regression) of the data and the exercise data is reliable for determining the exercise parameter if the error is higher than an error threshold.

The exercise data may be used to determine an estimation of the exercise parameter if the result “the criterion set is met by the judgement parameter set” is YES (step 23). The exercise parameter may be calculated based on the exercise data. Specifically, the exercise data may comprise a first portion of the exercise data that meets the criterion set 24 (i.e. the judgement parameter set 25 determined based on a first portion of the exercise data meets the criterion set 24) and a second portion of the exercise data which doesn't meet the criterion set 24 (i.e. the judgement parameter set 25 determined based on a second portion of the exercise data doesn't meet the criterion set 24); the exercise parameter can be calculated based on a first portion of the exercise data which meets the criterion set 24 (not based on a second portion of the exercise data which doesn't meet the criterion set 24). The exercise parameter may be calculated based on at least one of the internal workload data and the external workload data. In a first example, the exercise parameter may be calculated based on the internal workload data; in a second example, the exercise parameter may be calculated based on the external workload data; in a third example, the exercise parameter may be calculated based on a combination of the internal workload data and the first external workload data. On the contrary, the exercise data may not be used to determine an estimation of the exercise parameter if the result “the criterion set is met by the judgement parameter set” is NO.

Determining an estimation of the exercise parameter may comprise (1) calculating the exercise parameter based on at least one of the internal workload data and the external workload data after confirming that the criterion set 24 is met by the judgement parameter set 25 (i.e., the result is YES in step 23); and (2) calculating the exercise parameter based on at least one of the internal workload data and the external workload data before confirming whether the criterion set 24 is met by a judgement parameter set 25 or not and then perform the exercise parameter calculation based on at least one of the internal workload data and the external workload data after confirming that the criterion set 24 is met by the judgement parameter set 25 (i.e. the result is YES in step 23). After determining the estimation of the exercise parameter, the estimation of the exercise parameter may be displayed by a displaying unit 14 or the estimation of the exercise parameter or may be processed to generate the next/advanced exercise parameter.

The exercise parameter in EMBODIMENT (B) may be an energy expenditure, a fitness performance level (the fitness performance level may include health-related fitness and sport/skill-related fitness, which can also be improved by engaging in physical activities or training, e.g., VO2max or FTP (Functional Threshold Power)), first lactate threshold (LT1), second lactate threshold (LT2), maximum heart rate (HRmax) or minimum heart rate (HRmin), training load, fatigue, training effect, recovery, stamina. The exercise parameter may be calculated by any suitable method. For example, stamina and energy expenditure may be determined by taking U.S. application Ser. No. 14/718,104, U.S. application Ser. No. 17/070,040, U.S. application Ser. No. 17/070,947 as the references; the maximum heart rate may be determined by taking U.S. application Ser. No. 17/376,146 as a reference; the fitness performance level (e.g., VO2max or FTP (Functional Threshold Power) may be determined based on the maximum heart activity parameter (e.g., maximum heart rate (HRmax)) by any suitable method, such as a combination of the maximum heart activity parameter and a statistics of the internal workload data and the external workload data.

The disclosure further provides a computer-readable storage medium for executing the method for determining an exercise parameter if the exercise data is reliable. The computer-readable storage medium is composed of a plurality of program instructions (for example, a setting program instruction and a deployment program instruction) embodied therein. These program instructions can be loaded and executed by the same to execute the method for determining an exercise parameter if the exercise data is reliable described above.

The above disclosure is related to the detailed technical contents and inventive features thereof. People skilled in the art may proceed with a variety of modifications and replacements based on the disclosures and suggestions as described without departing from the characteristics thereof. Nevertheless, although such modifications and replacements are not fully disclosed in the above descriptions, they have substantially been covered in the following claims as appended.

What is claimed is:

1. A method for determining an exercise parameter, the method comprising:
acquiring exercise data, by a sensing unit, in an exercise session, wherein the exercise data comprises (i) an internal workload data set that includes a first parameter associated with an exercise intensity and (ii) an external workload data set that includes a second parameter associated with the exercise intensity, wherein the internal workload data set comprises a first internal workload data subset in a first duration of the exercise session and the external workload data set comprises a first external workload data subset in the first duration of the exercise session, wherein a variance of one of at least one of the first internal workload data subset and the first external workload data subset is higher than a first variance threshold;
confirming, by a processing unit, whether a criterion set is met by a judgement parameter set associated with a reliability metric in an estimation of the exercise parameter or not, wherein the judgement parameter set is determined based on a first feature parameter having consistency between a first trend of the first internal workload data subset and a second trend of the first external workload data subset; and
determining, by the processing unit or another processing unit, the estimation of the exercise parameter calculated based on at least one of the first internal workload data subset and the first external workload data subset if the criterion set is met by the judgement parameter set.

2. The method according to claim 1, wherein the judgement parameter set is determined further based on a second feature parameter being an extent to which the first internal workload data subset follows the first external workload data subset.

3. The method according to claim 2, wherein the judgement parameter set is determined further based on a third feature parameter being a duration length of the first duration when the first internal workload data subset and the first external workload data subset are acquired.

4. The method according to claim 1, wherein the judgement parameter set comprises a first judgement parameter associated with the reliability in an estimation of the exercise parameter, and the criterion set comprises a first criterion that describes that the first judgement parameter is higher than a reliability threshold, wherein the reliability in the estimation of the exercise parameter is determined based on a first feature parameter.

5. The method according to claim 4, wherein the reliability in the estimation of the exercise parameter is determined further based on a second feature parameter being an extent to which the first internal workload data subset workload data follows the first external workload data subset.

6. The method according to claim 5, wherein the internal workload data set further comprises a second internal workload data subset in a second duration of the exercise session and the external workload data set comprises a second external workload data subset in the second duration of the exercise session, wherein a variance of one of at least one of the first internal workload data subset and the first external workload data subset is higher than a variance threshold, wherein a second variance of one of at least one of the second internal workload data subset and the second external workload data subset is less than a second variance threshold, wherein the reliability in the estimation of the exercise parameter is determined further based on a third feature parameter being associated with the second internal workload data subset and the second external workload data subset.

7. The method according to claim 1, wherein the judgement parameter set comprises a first judgement parameter being the first feature parameter and the criterion set comprises a first criterion describing that the first judgement parameter is higher than a consistency threshold.

8. The method according to claim 7, wherein the judgement parameter set comprises a second judgement parameter, and the criterion set further comprises a second criterion that describes that the second judgement parameter is higher than an extent threshold, wherein the second feature parameter is an extent to which the first internal workload data subset follows the first external workload data subset.

9. The method according to claim 1, wherein the first parameter of the exercise intensity comprises a heart rate, an oxygen consumption, a pulse or a respiration rate, and wherein the second parameter of the exercise intensity comprises a speed, an acceleration, a power, an energy expenditure rate, or a motion cadence.

10. The method according to claim 1, wherein each of the first trend of the first internal workload data subset and the second trend of the first external workload data subset is an increasing trend of the corresponding exercise intensity varying with time or a decreasing trend of the corresponding exercise intensity varying with time.

11. The method according to claim 1, wherein the first external workload data subset is determined by modifying first initial internal workload data subset such that the first external workload data subset synchronizes with the first internal workload data subset higher than the first initial internal workload data subset.

12. The method according to claim 1, wherein the exercise parameter is a fitness performance level or an energy expenditure, and wherein the fitness performance level comprising VO₂ₘₐₓ or Functional Threshold Power (FTP).

13. The method according to claim 1, further comprising displaying, by a displaying unit, the estimation of the exercise parameter.

14. The method according to claim 1, wherein the judgement parameter set comprises a first judgement parameter being the first parameter of the exercise intensity, and the criterion set comprising a first criterion describing that the first judgement parameter is higher than a first intensity threshold, wherein the first intensity threshold is associated with a first history record of the first parameter of the exercise intensity.

15. The method according to claim 14, wherein the first intensity threshold is determined based on a first statistic of the first parameter of the exercise intensity.

16. The method according to claim 15, wherein the first statistic of the first parameter of the exercise intensity is a mean value of the first parameter of the exercise intensity.

17. The method according to claim 16, wherein the judgement parameter set comprises a second judgement parameter being the second parameter of the exercise intensity, and the criterion set comprises a second criterion that describes that the second judgement parameter is higher than a second intensity threshold, wherein the second intensity threshold is associated with a second history record of the second parameter of the exercise intensity

18. The method according to claim 17, wherein the judgement parameter set comprises a third judgement parameter determined based on a first feature parameter being a deviation degree between the internal workload data and the external workload data.

19. The method according to claim 18, wherein the third judgement parameter is the deviation degree between the internal workload data and the external workload data and the criterion set comprises a comparison between the third judgement parameter and a deviation threshold of the third judgement parameter.

20. A non-transitory computer-readable storage medium, the computer-readable storage medium recording an executable computer program, the executable computer program being loaded by an electronic device to:
acquire exercise data, by a sensing unit, in an exercise session, wherein the exercise data comprises (i) an internal workload data set that includes a first parameter of an exercise intensity and (ii) an external workload data set that includes a second parameter of the exercise intensity, wherein the internal workload data set comprises a first internal workload data subset in a first duration of the exercise session, and the external workload data set comprises a first external workload data subset in the first duration of the exercise session, wherein a variance of one of at least one of the first internal workload data subset and the first external workload data subset is higher than a first variance threshold;
confirm, by a processing unit, whether a criterion set is met by a judgement parameter set associated with a reliability in an estimation of the exercise parameter or not, wherein the judgement parameter set is determined based on a first feature parameter being a consistency between a first trend of the first internal workload data subset and a second trend of the first external workload data subset; and
determine, by the processing unit or another processing unit, the estimation of the exercise parameter calculated based on at least one of the first internal workload data subset and the first external workload data subset if the criterion set is met by the judgement parameter set.
